(19) United States
(12) Patent Application Publication
(10) Pub. No.: US 2004/0213742 A1
(43) Pub. Date: Oct. 28, 2004

(54) Monitoring marker

(76) Inventor: Robert Hahn, Tullinge (SE)

Correspondence Address: BROWDY AND NEIMARK, P.L.L.C., 624 NINTH STREET, NW, SUITE 300, WASHINGTON, DC, 20001-5303, US

(21) Appl. No.: 10/478,957

(22) PCT Filed: Mar. 26, 2003
(86) PCT No.: PCT/SE03/00504

(30) Foreign Application Priority Data
Mar. 27, 2002 (SE) 0200931-4

(51) Int. Cl. A61K 49/00

(52) U.S. Cl. 424/9.52

(57) ABSTRACT

Use of a gas or gas precursor for the manufacture of a monitoring agent for a diagnostic method for monitoring fallopian tube abnormalities in a female mammal, including man, wherein said monitoring agent is diagnostically acceptable gas in the gaseous state or dissolved in a diagnostically acceptable liquid as a true solution, said gas being of such a nature, and being used in such an amount, that is detectable via the expired breath from the mammal in question. An especially preferable gas in nitrous oxide. Said use is preferably for the detection of infertility of the mammal.

### TECHNICAL FIELD

The present invention is within the field of diagnosing fallopian tube abnormality in a female mammal, including man. More specifically it relates to the use of a gas or gas precursor as a marker, which is detectable via the expired breath from said mammal. The invention is especially of great value in the assessment of female fertility.

### BACKGROUND OF THE INVENTION

The fallopian tubes project from each side of the uterus and from the passages through which the egg is conducted from the ovary into the uterus. Said tubes are approximately 10 cm long and fertilization occurs in the outer half thereof. As a result of the complex structure of the tubes tube abnormalities account for between 25% and 50% of female infertily. The reason for infertility can be malformation of the fallopian tubes but more often tubal damage occurs from pelvic infection (pelvic inflammatory disease PID).

Therefore, when diagnosing infertility a test to determine whether or not the fallopian tubes are open is an important methodology.

The most frequently used test methods are based on X-ray or ultrasonic examinations, said examinations being made after passing a fluid or a contrast medium into the uterus under high pressure. Apart from being discomfortable to the patients such methods are also expensive and rather complicated, which means that in principle they have to be performed in a hospital.

U.S. Pat. No. 5,529,766; U.S. Ser. No. 2001/0019710 A1 and WO 94/09703 are examples of prior art based on ultrasonic examinations. More specifically, U.S. Pat. No. 5,529,766 discloses ultrasound contrast agents comprising microbubbles of gas or gas precursor encapsulated in a shell of protein crosslinked with biodegradable crosslinking groups, while U.S. Ser. No. 2001/0019710 A1 discloses contrast agents in the form of oil-in-water emulsions and WO 94/09703 discloses contrast agents comprising gas microbubbles, which are stabilized by means of a combination of surfactants.

Common to all of these prior art documents is that the contrast action of these agents is due to gas bubbles, which technique is, as was said above, discomfortable, expensive and rather complicated.

Even more sophisticated techniques are based on the insertion of special catheters or fiberoptic tubes into the fallopian tubes to enable visualizations thereof.

Furthermore, common for all known test methods thus is that they are rather complicated and require highly competent and trained persons for the performances thereof.

The present invention is based on a completely new and simple technique for performing a test of the above-mentioned kind.

### DISCLOSURE OF THE INVENTION

The present invention is primarily based on the finding that when administering a gas to the uterus of a woman said gas is detectable via the expired breath from said woman in normal cases. However, in the case of fallopian tube abnormalities, especially obstructions, the passage of gas from the uterus into the fallopian tubes is reduced or even blocked, such disturbances being thus detectable in the expired breath.

When a gas is used in this way as a detectable marker, already small amounts thereof can be detected rapidly. In other words, the present invention enables the use of a completely new technique for monitoring fallopian tube abnormalities, which technique does not cause any discomfort to the woman in question.

In addition thereto, the new technique is very simple, as there will for instance not be needed any visual determinations by highly competent or trained personal. It would also be possible to use the same polyclinically and the low costs thereof would also be an advantage, e.g. for use in developing countries.

Furthermore, the gas can be added to a carrier fluid in advance and kept therein, e.g. in solution for several years, if using a suitable container material. In addition thereto, detection of exhaled gas can be very accurate and can be made with simple and even existing monitors. For instance, in the case of nitrous oxide (N₂O) such monitors are available in anaesthesia machines, which monitors may be used as such or easily converted into more accurate monitors.

Other purposes or advantages with the invention should be apparent to a person skilled in the art after having read the description below.

More specifically, according to a first aspect of the present invention, there is provided use of a gas or gas precursor for the manufacture of a monitoring agent for a diagnostic method for monitoring fallopian tube abnormalities in a female mammal, including man, wherein said monitoring agent is a diagnostically acceptable gas in the gaseous state or a diagnostically gas dissolved in a diagnostically acceptable liquid as a true solution, said gas being of such a nature and being used in such an amount, that it is detectable via the expired breath from the mammal in question.

In other words, in addition to a gas which is detectable via the expired breath from a mammal, the invention is applicable also the use of a gas precursor, i.e. a substance that is the source of such a gas in connection with the diagnostic method referred to.

Both the gas and the gas precursor should be diagnostically acceptable.

The gas precursor is preferably a volatile liquid, i.e. a liquid which is readily vaporizable at a relatively low temperature, e.g. below 70° C. or even below 40° C. or 30° C.

The term fallopian tube abnormalities as used in connection with the present invention generally means obstructions of any kind of the fallopian tubes. Especially, the monitoring is for the detection of infertility of the mammal in question, since generally to accomplish pregnancy for a woman at least one of her fallopian tubes, or salpinges, has to be open, or permeable, for fluids.

The gas or gas precursor can be used as such but it is also possible to use a diagnostically acceptable liquid as a carrier medium, said gas or gas precursor being dissolved therein. The carrier liquid could be any diagnostically acceptable liquid, provided that it does not cause any disturbances on the detection of the gas. Generally, however, the monitoring fluid should be an isotonic solution, preferably physiological saline.

The term “used as such” means that the gas or gas precursor is used in its gaseous state. This does not necessarily mean, however, that said gas or gas precursor is used alone. Gas or gas precursor in a carrier gas or mixtures thereof may also be utilized.

In the case of using the gas dissolved in a liquid it is important to note that this refers to a real or true solution of the gas in said liquid as opposed to the prior art where gas bubbles are detected by e.g. ultrasonic imaging.

In accordance with the present invention the gas is detectable in the expired breath from the mammal in question. Accordingly, the gas is present in the monitoring agent in such an amount or concentration that is detectable in the expired breath from the mammal by the desired gas monitor.

One embodiment of the invention in this respect is represented by the case where said gas has been dissolved to its saturation level in said liquid, generally at normal pressure and ambient (room) temperature.

In other words, the concentration of gas is generally between the lowest detectable concentration up to said saturation concentration. In the case of using nitrous oxide as said gas, the concentration can e.g. be from 0.01 to 0.65 L of gaseous nitrous oxide per L of liquid, preferably from 0.01 to 0.5 L, more preferably from 0.05 to 0.5 L, especially from 0.05 to 0.3 L. For other gases corresponding or similar concentrations can easily be calculated.

The gas or gas precursor is preferably mixed with or dissolved in the carrier and stored together with said carrier up to the use thereof in the diagnostic method to be performed. However, it is of course also within the scope of protection to feed or add the gas or gas precursor separately in connection with the diagnostic method, as in general similar effects should be obtainable by any of these methods.

When used as such the gas or gas precursor is typically used in an amount of 0.1-10 mL, more specifically 1-5 mL, calculated as the pure gas, especially in the case of using N₂O. However, the optimum amount is easily determined by a person skilled in the art in each specific case.

Expressed in another way it could be said that such amounts of gas, in the gaseous state or dissolved in a liquid, are used that a detection within the range of 1-1000 ppm of gas is achieved.

According to still another preferable embodiment of the use according to the invention the monitoring agent is adapted to be administered to the uterus of the female in question.

Although generally the characteristics of the gas or gas precursor can not be specified in exact figures, it should be easy for a person skilled in the art to screen candidates for gases or gas precursors without undue experimental work, as long as the gas or gas precursor is diagnostically acceptable and is of such a nature that it is detectable in the expired breath from the mammal in question. Giudance in this respect follows from the following examples of useful gases or gas precursors:

Nitrous oxide (N₂O):

noble gases, e.g. argon, krypton and xenon;

lower hydrocarbons, e.g. ethane, ethene and acetylene;

sulphur hexafluoride (SF₂); and

fluorinated lower hydrocarbons or hydrocarbon derivatives, e.g. volatile inhalation anesthetics such as sevoflurane (=fluoromethyl-2,2,2-trifluoro-1-(trifluoromethyl)ethyl ether.

Nitrous oxide is especially preferable.

According to a second aspect of the invention, or expressed in another way, there is also provided a monitoring agent for a diagnostic method for monitoring fallopian tube abnormalities in a female mammal, including man, which is a diagnostically acceptable gas in the gaseous state or a diagnostically acceptable gas dissolved in a diagnostically acceptable liquid as a true solution, said gas being of such a nature, and being used in such an amount, that it is detectable via the expired breath from the mammal in question.

As to preferable embodiments of said monitoring agent reference is made to the those preferable embodiments which have been presented in connection with the use described above. Thus, such preferable embodiments should be applicable also to the monitoring agent per se.

Still another aspect of the invention is represented by a method of monitoring fallopian tube abnormalities in a female mammal, including man, which comprises administering to the uterus of said mammal a monitoring agent as defined above and monitoring the expired breath from said mammal by means of a gas detector to detect the gas therein.

Also in this case all preferable embodiments are applicable which have been described above in connection with the use.

The gas monitoring device, or detector, to be used in connection with the present invention could be selected among previously known gas detectors used in other connections or easily modified therefrom. As was said above, the device referred to should be able to operate at least within the range of 1-1000 ppm.

The detection is made as a relative measuring of the gas in comparison with the amount of gas which is detectable in the “normal” case, i.e. when the passage between uterus into the fallopian tubes is not blocked.

Said detection may be made in an end-expiratory manner but generally this is not necessary. Thus, it can be made any time in the respiratory cycle.

### FIGURES

The accompanying figures show the following:

FIG. 1 represents a graph showing the concentration of N₂O versus time in expired air after intrauterine administration thereof, for a case where the uterine tubes are open compared to a case where they are closed; and

FIG. 2 represents a graph showing the cumulative N₂O concentrations versus time for the experiment shown in FIG. 1.

### EXAMPLE

The invention will now be further described in a non-limiting way by the following working example.

Pure nitrous oxide (N₂O) gas was injected into the uterine cavities of 2 anaesthetized pigs with open uterine tubes and surgically ligated uterine tubes, respectively. Said N₂O was detected every second in the expired breath by means of a conventional N₂O detector.

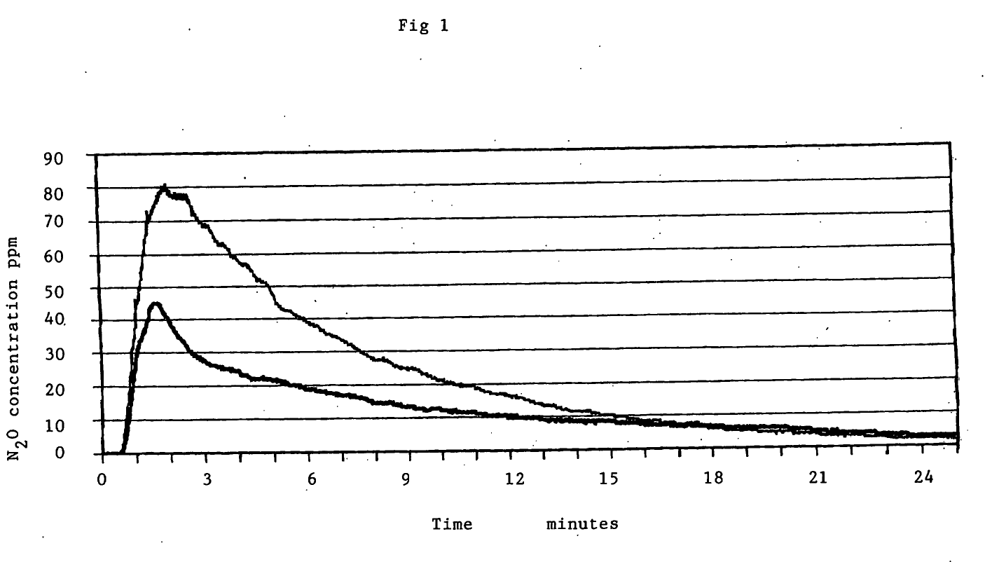

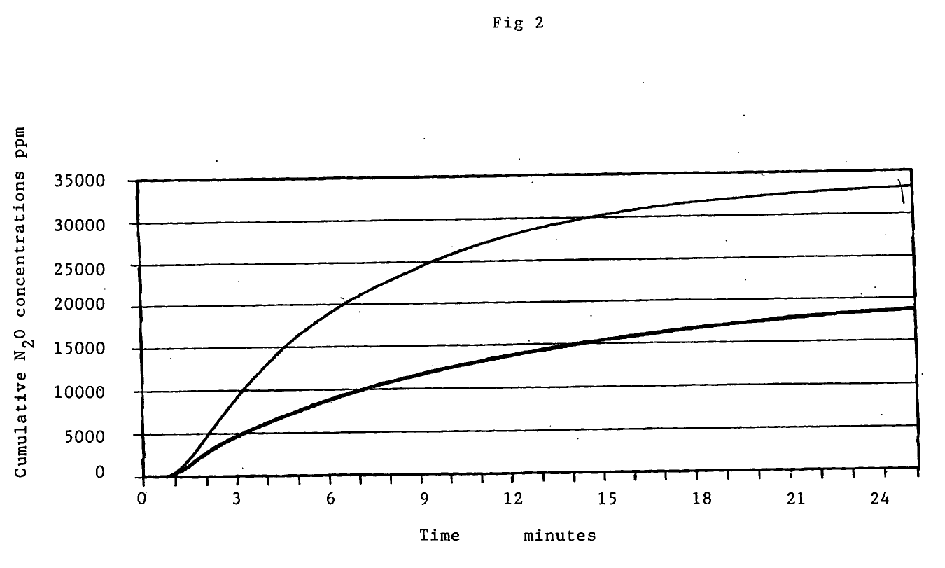

The results are shown in FIGS. 1 and 2, where FIG. 1 is a concentration versus time graph and FIG. 2 is a cumulative concentration versus time graph, the upper curves representing closed uterine tubes and the lower curves representing open uterine tubes.

As can be seen from said Figures a maximum concentration in the exhaled air of approximately 40 ppm was activated after approximately 2 minutes in the case of open uterine tubes while said value was approximately 50% higher in the case of closed tubes. Furthermore, there was a clear difference between the two curves versus time which enables a monitoring of fallopian tube abnormalities as claimed by the present invention.

1-24. (canceled)

25. A method of monitoring fallopian tube abnormalities in female mammal, including man, which comprises
administering to said mammal, as a monitoring agent, a diagnostically acceptable gas in the gaseous state or a diagnostically acceptable gas dissolved in a diagnostically acceptable liquid as a true solution
said gas being of such a nature, and being used in such an amount, that it is
detectable via the expired breath from the mammal in question; and
monitoring the expired breath from said mammal with a gas detector to detect the gas therein.

26. A method according to claim 25, wherein said administering is to the uterus of said mammal.

27. A method according to claim 25, wherein said monitoring is for the detection of fallopian tube obstruction.

28. A method according to claim 25, wherein said monitoring is for the detection of infertility of said mammal.

29. A method according to claim 25, wherein said gas is detected quantitatively.

30. A method according to claim 25, wherein said monitoring agent is a diagnostically acceptable gas in the gaseous state.

31. A method according to claim 25, wherein said liquid is an isotonic liquid solution.

32. A method according to claim 31, wherein said isotonic solution is physiological saline.

33. A method according to claim 25, wherein said gas has been dissolved to its saturation level in said liquid.

34. A method according to claim 25, wherein said gas is nitrous oxide.

35. A method according to claim 25, wherein said gas is a noble gas.

36. A method according to claim 35, wherein said noble gas is selected from the group consisting of argon, krypton and xenon.

37. A method according to claim 25, wherein said gas is a lower hydrocarbon.

38. A method according to claim 25, wherein said lower hydrocarbon is selected from the group consisting of ethane, ethene and acetylene.

39. A method according to claim 25, wherein said gas is sulphur hexafluoride.

40-42. (canceled)

43. A monitoring agent for a diagnostic method for monitoring fallopian tube abnormalities in a female mammal, including human, which is a diagnostically acceptable gas in the gaseous state or a diagnostically acceptable gas dissolved in a diagnostically acceptable liquid as a true solution, said gas being of such a nature, and being used in such an amount, that it is detectable via the expired breath from the mammal in question.

44. A monitoring agent according to claim 43, wherein said liquid is an isotonic liquid solution.

45. A monitoring agent according to claim 44, wherein said isotonic solution is physiological saline.

46. A monitoring agent according to claim 43, wherein said gas has been dissolved to its saturation level in said liquid.

47. A monitoring agent according to claim 43, wherein said gas is nitrous oxide.

48. A monitoring agent according to claim 43, wherein said gas is a noble gas.

49. A monitoring agent according to claim 48, wherein said noble gas is selected from the group consisting of argon, krypton and xenon.

50. A monitoring agent according to claim 43, wherein said gas is a lower hydrocarbon.

51. A monitoring agent according to claim 50, wherein said lower hydrocarbon is selected from the group consisting of ethane, ethene and acetylene.

52. A monitoring agent according to claim 43, wherein said gas is sulphur hexafluoride.

53-55. (canceled)

56. A method according to claim 25, wherein said diagnostically acceptable gas is derived from a gas precursor.

57. A method according to claim 56, wherein said gas precursor is a liquid.

58. A method according to claim 57, wherein said liquid is a fluorinated lower hydrocarbon or hydrocarbon derivative.

59. A method according to claim 58, wherein said fluorinated lower hydrocarbon or hydrocarbon derivative is sevoflurane.

60. A monitoring agent according to claim 43, wherein said diagnostically acceptable gas is from a gas precursor.

61. A monitoring agent according to claim 60, wherein said gas precursor is a liquid.

62. A monitoring agent according to claim 61, wherein said liquid is a fluorinated lower hydrocarbon or hydrocarbon derivative.

63. A monitoring agent according to claim 62, wherein said fluorinated lower hydrocarbon or hydrocarbon derivative is sevoflurane.
